(19) United States
(12) Patent Application Publication
(10) Pub. No.: US 2024/0189277 A1
(43) Pub. Date: Jun. 13, 2024

(54) OPHTHALMIC FORMULATIONS FOR SUSTAINED NEUROPROTECTION

(71) Applicant: The Johns Hopkins University, Baltimore, MD (US)

(72) Inventors: Laura Ensign, Baltimore, MD (US); Justin Hanes, Baltimore, MD (US); Yoo Chun Kim, Baltimore, MD (US); Tung Heng Hsueh, Baltimore, MD (US)

(21) Appl. No.: 18/554,928

(22) PCT Filed: Apr. 14, 2022
(86) PCT No.: PCT/US2022/024826

§ 371 Date: Oct. 11, 2023

Related U.S. Application Data
(60) Provisional application No. 63/175,621, filed on Apr. 16, 2021.

(51) Int. Cl. A61K 31/404; A61K 9/00; A61K 31/192; A61P 27/02

(52) U.S. Cl. CPC A61K 31/404; A61K 9/0019; A61K 9/0048; A61K 31/192; A61P 27/02

(57) ABSTRACT

Administration of therapeutic, prophylactic, or diagnostic agents that bind to a receptor or ligand in the eye or to the skin in microcrystalline form leads to increased delivery and long-lasting activity within the eye as compared with administration of the agent in non-crystalline form. Compositions and methods for delivery of active agents in microcrystalline form to the eye for the treatment, prevention and diagnosis of eye diseases and disorders are provided. In preferred embodiments, microcrystals include sunitinib malate in complex with pamoic acid. Administration of microcrystals of sunitinib malate/pamoic acid to the eye provides protection from glaucoma for up to 6 months or longer without retinal toxicity, following a single injection into the eye.

### CROSS-REFERENCE TO RELATED APPLICATIONS

This application claims priority to and benefit of U.S. Provisional Application No. 63/175,621, filed Apr. 16, 2021, which is hereby incorporated herein by reference in its entirety.

### STATEMENT REGARDING FEDERALLY SPONSORED RESEARCH

This invention was made with government support under grants EB016121, EY026578, and EY031041 awarded by the National Institutes of Health. The government has certain rights in the invention.

### FIELD OF THE INVENTION

The present invention is in the field of extended-release formulations for ophthalmic administration, and more particularly in the area of injectable formulations of drug microcrystals for sustained drug delivery in the eye.

### BACKGROUND OF THE INVENTION

Glaucoma is the leading cause of irreversible blindness, impacting 80 million people worldwide (Quigley, H. A. and Broman, A. T., Br J Ophthalmol, 2006. 90(3): p. 262-7; Fundation, B. Glaucoma: Facts & Figures. 2021 Mar. 7, 2021). Due to the rapid increase in the aging population, the number is expected to rise to 111 million by 2040 (Tham, Y. C., et al., Ophthalmology, 2014. 121(11): p. 2081-2090). Glaucoma is a neurodegenerative disease that causes irreversible vision loss due to the death of retinal ganglion cells (RGCs). All of the current standard-of-care treatments for glaucoma are directed to lowering intraocular pressure (IOP). However, maintaining lower pressure can be challenging in some patients, and in others, even if significant pressure lowering is achieved, RGC loss continues (Quigley, H. A., Lancet, 2011. 377(9774): p. 1367-1377).

Dual leucine zipper kinase (DLK; MAP3K12) is a key mediator of RGC cell death in response in axonal injury (Welsbie, D. S., et al., Proc Natl Acad Sci USA, 2013. 110(10): p. 4045-50; Welsbie, D. S., et al., Neuron, 2017. 94(6): p. 1142-1154 e6). Compounds with activity against DLK that potently promote survival of mouse and human RGCs have been identified, including the FDA-approved kinase inhibitor sunitinib (Welsbie, D. S., et al., Neuron, 2017. 94(6): p. 1142-1154 e6). Development of sunitinib into a formulation for effective, sustained intraocular delivery as a complement to IOP-lowering strategies could improve glaucoma treatment and management (Chader, G. J., Invest Ophthalmol Vis Sci, 2012. 53(5): p. 2501-5; Chang, E. E. and J. L. Goldberg, Ophthalmology, 2012. 119(5): p. 979-86; Danesh-Meyer, H. V., Curr Opin Ophthalmol, 2011. 22(2): p. 78-86; Hanumunthadu, D., M. H. Dehabadi, and M. F. Cordeiro, Expert Review of Ophthalmology, 2014. 9(2): p. 109-123).

Patient adherence is critical for treating chronic diseases like glaucoma, though administration difficulties and side effects associated with glaucoma eye drop treatments result in high levels of non-compliance (Kaufman. and Rasmussen, Ophthalmol Vis Sci, 2012. 53(5): p. 2495-500). Typically, only 40-60% of patients adhere to glaucoma drop therapy regimens, even in cases where the patients were being monitored and were provided free medication (Nordstrom, B. L., et al., Am J Ophthalmol, 2005. 140(4): p. 598-606; Okeke, C. O., et al., Ophthalmology, 2009. 116(12): p. 2286-93; Okeke, C. O., et al., Ophthalmology, 2009. 116(2): p. 191-9).

Achieving effective drug delivery to the posterior segment with an eye drop is challenging (Rodrigues, G. A., et al., Pharm Res, 2018. 35(12): p. 245; Singh, R., et al., Retina, 2014. 34(9): p. 1787-95; Nomoto, H., et al., Invest Ophthalmol Vis Sci, 2009. 50(10): p. 4807-13; Iwase, T., et al., Invest Ophthalmol Vis Sci, 2013. 54(1): p. 503-11). The most common approach for achieving drug delivery to the posterior segment is intravitreal (IVT) injection, which is employed every 6-8 weeks to deliver biologics for treatment of wet age-related macular degeneration (Avery, R. L., et al., Ophthalmology, 2006. 113(3): p. 363-372 e5; Becerra, E. M., et al., Curr Drug Targets, 2011. 12(2): p. 149-72). However, IVT injection is relatively invasive, and is associated with rare, but severe ocular complications (Jager, R. D., et al., Retina, 2004. 24(5): p. 676-98; Boyer, D. S., et al., Ophthalmology, 2014. 121(10): p. 1904-14). For a disease that may require treatment for decades, a less invasive injection approach is preferred. Further, longer times between injections would be preferred by glaucoma patients, particularly since it may take years to notice deterioration of vision.

Therefore, it is an object of the invention to provide formulations for the effective and sustained intraocular delivery of active agents to treat or alleviate one or more symptoms of glaucoma.

It is a further object of the present invention to provide formulations for the effective and sustained delivery of active agents to the posterior segment of the eye.

### SUMMARY OF THE INVENTION

Microcrystals formed of active agents that bind to a receptor or ligand in the eye leads to increased delivery and long-lasting efficacy of the active agent within the eye. Injectable formulations for administration into the eye include:
(a) one or more active agents which bind to a receptor or ligand in the eye, in the form of microcrystals; and
(b) one or more pharmaceutically acceptable excipients for administration to the eye.

An exemplary therapeutic agent is an inhibitor of dual leucine zipper kinase (DLK). In preferred embodiments, the active agents bind to a ligand such as melanin within the eye with a dissociation constant (K_{d}) of less than 1×10⁻³ M, 1×10⁴ M, 1×10⁻⁵ M, or 1×10⁻⁶ M. Preferred agents include sunitinib, acriflavine, or a derivative, analogue, or prodrug thereof. In one preferred embodiment, the microcrystals are formed of a complex between sunitinib malate and pamoic acid.

Typically, the size of the microcrystals is between about 2 microns and about 200 microns, preferably between about 5 microns and about 100 microns, and the pH of the formulation is between about pH 6 and about pH 8. In some embodiments, the microcrystals are formulated for injection, for example, by intravitreal, subconjunctival, or suprachoroidal administration. In some embodiments, the formulation does not contain a preservative or surfactant. The volume of a microcrystal formulation is typically selected according to the method of administration and site of administration.

Methods of administering microcrystals of active agents for preventing, treating or diagnosing one or more diseases, conditions, or injuries of the eye in a subject are also provided. In some embodiments, the methods include administering to the eye of the subject a therapeutically effective amount of a formulation including
(a) one or more active agents, preferably binding to a receptor or ligand within the eye, in the form of microcrystals; and
(b) one or more pharmaceutically acceptable excipients for administration to the eye.

In some embodiments, the compositions include agents which are effective to treat or prevent one or more diseases, conditions, and injuries of the posterior segment of the eye. Exemplary eye diseases, conditions and injuries that may be treated and/or prevented by the compositions include diseases of the retina, choroid, and/or optic nerve, eye injuries such as retinal tear and retinal detachment, diabetic retinopathy, epiretinal membrane, macular hole, macular degeneration, retinitis pigmentosa, retinal neovascularization, and choroid neovascularization.

In some embodiments, the formulations are administered to the choroid and/or retinal pigmented epithelium (RPE) in the eye. In particular embodiments, the methods deliver microcrystals of active agents to the eye to treat and prevent retinal and/or choroidal neovascularization. In some embodiments, the amount of active agent(s) administered to the eye in the form of microcrystals is effective to reduce retinal and/or choroidal neovascularization to a greater extent than the equivalent amount of the same active agent(s) delivered without microcrystal formulation. For example, in some embodiments, the methods reduce retinal and/or choroidal neovascularization by 10%, 20%, 30%, 40%, 50%, or more than 50% relative to the equivalent amount of the same active agent(s) delivered in a form other than as microcrystals. In other embodiments, the amount of active agent(s) effective to reduce retinal and/or choroidal neovascularization is 10%, 20%, 30%, 40%, 50%, or more than 50% less than the amount of the same active agent(s) that is required without microcrystal formulation. Typically, the microcrystal formulations are administered into the eye by injection, for example, via intra-vitreal, sub-conjunctival, or supra-choroidal injection. Microcrystals administered by injection into the eye in a low volume, such as between about 0.1 μl and about 100 μl, inclusive, provides optimal long-term administration following a single injection into the eye. Therefore, in some embodiments, the volume of microcrystal formulations is between about 0.1 μl and about 100 μl, preferably between about 1 μl and 10 μl.

In some embodiments, the formulations of microcrystals are administered once a week or less frequently. Typically, the methods provide therapeutic or prophylactic efficacy in the subject for a period of time such as one week, two weeks, three weeks, one month, two months, three months, four months, five months, six months, or one year, following a single administration to the eye.

In a preferred embodiment, microcrystals include sunitinib malate in complex with pamoic acid. A single injection into the eye of microcrystals of sunitinib malate/pamoic acid to the eye provides protection from glaucoma for up to 6 months or longer without retinal toxicity.

Methods for making crystalline formulations of active agents are also described.

### BRIEF DESCRIPTION OF THE DRAWINGS

FIG. 1A is a chart showing solubility of the sunitinib-pamoate complex (SPC) in organic solvents including acetone, ethanol and diethyl ether mixed with water at the volume ratios shown on the x-axis. FIG. 1B is a chart showing the outcome of stirring solutions of SPC dissolved in ethanol/water solutions at various temperatures (refrigeration, 4° C.; room temperature, 25° C.; water bath, 37° C.) for 2 hours. Outcomes included remaining as a solution, forming aggregates that adhered to the container wall and stir bar, and formation of microcrystals.

FIG. 2A is a bar graph showing complexation efficiency of sunitinib malate when mixed at various ratios of pamoic acid to sunitinib (1-20 w:w, n=3). Data shown as mean±SD. FIG. 2B is a bar graph showing size (μm) of SPC microcrystals over a period of 180 days at room temperature (n=3). Data shown as mean±SD.

FIG. 3 is a bar graph showing number of viable RGCs in vitro versus concentration of Sodium Pamoate (mg/ml). Primary mouse cells were plated and treated with 1 μM (or 398 ng/mL) sunitinib malate to provide neuroprotection. RGCs were treated with either sodium pamoate or PBS to assess the possible interference in the neuroprotective effect of sunitinib. After 72 h, survival of the cells was analyzed using viability assay (n=3-5). Data represented as the mean±SEM.

FIG. 4A is a bar graph showing combined levels of sunitinib and N-desethyl sunitinib in retina tissues (measured in ng/g) 7 days after a single unilateral subconjunctival injection of different doses of SPC microcrystals in 5 μL volume was given to rats. n=5-6. Data shown as median ±IQR. *p<0.05 compared to all other doses. FIG. 4B is a bar graph showing combined levels of sunitinib and N-desethyl sunitinib in retina tissues (measured in ng/g) 7 days after a single unilateral subconjunctival injection of 200 μg SPC microcrystals was given to rats in two different injection volumes (5 μL and 100 μL). n=5. Data shown as median ±IQR. **p<0.01. FIG. 4C is a bar graph showing combined levels of sunitinib and N-desethyl sunitinib in retina tissues (measured in ng/g) 7 days after a single unilateral subconjunctival injection of 200 μg SPC microcrystals was given to rabbits in two different injection volumes (5 μL and 100 μL). n=4-5. Data shown as median ±IQR. **p<0.01.

FIG. 5A is a line graph showing combined sunitinib and N-desethyl sunitinib levels (ng/g) in the retina over a period of 20 weeks after a single unilateral subconjunctival injection of SPC microcrystals (200 μg in 5 μL) was given in rats. n=4-9. Data shown as median ±IQR. FIG. 5B is a bar graph showing percent survival of RGCs in whole retinal flat mounts in groups without treatment (Crush Only) and with a single injection of SPC in subconjunctival space at 3, 8, and 20 weeks post injection. SPC was injected unilaterally at t=0 wk and optic nerve crush was performed 2 weeks before the indicated time points (n=6-12). Data shown as mean±SEM. *p<0.05. Statistical analyses conducted by one-way ANOVA with multiple comparisons.

FIG. 6A is a line graph showing combined concentration of sunitinib and N-Desethyl sunitinib in the choroid/retinal pigmented epithelium (Ch/RPE) over a period of 20 weeks after a single administration of SPC (200 μg) into the subconjunctival space (n=4-9). Data shown as median ±interquartile range. FIG. 6B is a bar graph showing combined concentration of sunitinib and N-Desethyl sunitinib in the choroid/retinal pigmented epithelium (Ch/RPE) at 7 days after a single unilateral administration of SPC microcrystals at a dose of either 200 μg (in 5 μl) or 20 mg (in 50 μL).

FIG. 7A is a bar graph showing combined levels of sunitinib and N-desethyl sunitinib in cornea tissue 7 days after a single unilateral subconjunctival injection of 100 μg SPC microcrystals was given to rats in two different injection volumes (5 μL and 100 μL). FIG. 7B is a bar graph showing area of neovascularization (NV) in mm² one week after SPC microcrystals (100 μg in 5 μL) were injected unilaterally at the same time as suturing the cornea to induce neovascularization, compared to untreated control. n=4-8. Data shown as mean±SD. *p<0.05. Statistical analyses conducted by Student's t-test.

### DETAILED DESCRIPTION OF THE INVENTION

### I. Definitions

The term “microcrystal” of an active agent refers to a crystalline form of the agent, typically a synthetic or natural compound having a molecular weight of less than 2000D, more typically less than 1500 or 1000 D, having dimensions between about 0.1 μm and about 1,000 μm, inclusive. Microcrystals can be either crystalline, amorphous, or a mixture thereof. The term “microcrystal formulation” infers that at least one active agent be present in the formulation is in crystalline form. Typically, the concentration of active agent within a microcrystal is greater than the maximum concentration of active agent in solution. In some embodiments, the concentration of active agent within a microcrystal is between about 1 and about 1,000 mg/ml.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “biocompatible” and “biologically compatible” generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

The terms “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated a disease or disorder of the eye is mitigated or eliminated, including, but not limited to, reducing vision loss, improving visual acuity, increasing the expression of γ-synuclein and/or βIII tubulin in retinal ganglion cells following optic nerve injury, lowering intra-ocular pressure, reducing vascular leakage from retinal blood vessels, and reducing retinal and/or choroidal neovascularization.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effect of the effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

“Excipient” is used herein to include a compound that is not a therapeutically or biologically active compound. As such, an excipient should generally be inert and non-toxic to the subject.

The term “analog” refers to a chemical compound with a structure similar to that of another “reference” compound, but differing from it in respect to a particular component, functional group, atom, etc.

The term “derivative” refers to a compound, which is formed from a parent compound by one or more chemical reaction(s).

### II. Compositions

Compositions including therapeutic, prophylactic and/or diagnostic agents in micro-crystalline form, particular those binding to a receptor or ligand in the eye, enhance and prolong the therapeutic, prophylactic, or diagnostic efficacy of the agents within the eye as compared with administration of the agent in non-crystalline form. Compositions of microcrystals of agents for the treatment, prevention and diagnosis of eye diseases and disorders are provided. In preferred embodiments, microcrystals include sunitinib malate in complex with pamoic acid. Pharmaceutical compositions and dosage forms of microcrystals for administration to the eye are also described.

A. Therapeutic, Prophylactic and/or Diagnostic Agents

The compositions contain one or more agents to be delivered. Examples include therapeutic agents, prophylactic agents, and/or diagnostic agents. A biologically active agent is a substance used for the treatment (e.g., therapeutic agent), prevention (e.g., prophylactic agent), diagnosis (e.g., diagnostic agent), to effect or assist in the effecting of a cure or mitigation of a disease or defect. The agent may also be a pro-drug which becomes biologically active or more active when placed in a predetermined physiological environment.

Agents may be small-molecule drugs ((e.g., molecular weight less than 2000, 1500, 1000, 750, or 500 atomic mass units (amu)), peptides or proteins, sugars or polysaccharides, nucleotides or oligonucleotides such as aptamers, siRNA, and miRNA, lipids, glycoproteins, lipoproteins, or combinations thereof.

Agents may be described in Martindale: The Complete Drug Reference, 37^{th} Ed. (Pharmaceutical Press, London, 2011) or others known to those skilled in the art or described in the scientific literature. In one embodiment, the agent to be delivered is poorly soluble in water, and may be administered as a suspension or in a non-aqueous formulation. In other embodiments, the agents are water-soluble.

The formulations can contain a therapeutically effective amount of a therapeutic agent to treat, inhibit, or alleviate one or more symptoms of a disease state being treated. The compositions can contain an effective amount of a prophylactic agent to prevent one or more symptoms of a disease or disorder in the eye.

In preferred embodiments, the active agents are one or more small molecule drugs that are less than about 2000 g/mol in molecular weight, less than about 1500 g/mol, less than about 1000 g/mol, less than about 800 g/mol, or less than about 500 g/mol. In further preferred embodiments, the active agents are one or more small molecule drugs that bind to melanin.

Agents may be anti-infectives (antibiotics, antivirals, antifungals), for treatment of eye disorders (glaucoma, dry eye), anti-inflammatories, inhibitors of neovascularization, neuroactive agents, or chemotherapeutics for treatment of a disease such as cancer. Exemplary agents include brinzolamide, cyclosporine A, brimonidine tartrate, moxifloxacin, and budesonide.

In preferred embodiments, the active agent is an inhibitor of dual leucine zipper kinase (DLK). In further preferred embodiments, the active agent binds to melanin in the eye. Exemplary agents include sunitinib and acriflavine.

Representative anti-proliferative (anti-cancer or endometriosis) agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof. Other suitable anti-cancer agents include angiogenesis inhibitors including antibodies to vascular endothelial growth factor (VEGF), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

In preferred embodiments, the active agent is a small molecule tyrosine kinase inhibitor or a small molecule inhibitor of VEGF receptors.

For diagnostic applications, including imaging, radioactive materials such as Technetium99 (^{99m}Tc) or magnetic materials such as labelled-Fe₂O₃ could be used. Examples of other materials include compounds which are radiopaque.

In some embodiments, the one or more active agents bind to melanin, for example with a strong binding capacity (moles drug/mg melanin), preferably more than 1×10⁻⁸ M, 1×10⁻⁷ M, 1×10⁻⁶ M, or 1×10⁻⁵ M, and/or a low dissociation constant (K_{d}), preferably less than 1×10⁻³ M, 1×10⁴ M, 1×10⁻⁵ M, or 1×10⁻⁶ M.

In preferred embodiments, the one or more agents binding to melanin are efficiently taken up into melanin-producing cells at the target site. In further preferred embodiments, the compositions include one or more agents bind to melanin and one or more additional therapeutic agents, prophylactic agents, diagnostic agents, and/or nutraceuticals.

In a particular embodiment, the active agent is sunitinib, acriflavine, or a derivative, an analogue, and a prodrug thereof.

### B. Microcrystals of Active Agents

Compositions in the form of microcrystals are provided. The compositions can be in the orm of a liquid dispersion, or suspension or provided as a solid which is suspended or dispersed for administration.

Crystals of active agents can include one or more protein, nucleic acid, lipid, carbohydrate, salt, and small molecule active agents. In some embodiments, the crystals include two or more active agents, for example, as a complex. In preferred embodiments, the active agents are one or more small molecule drugs that are less than about 2000 g/mol in molecular weight, less than about 1500 g/mol, less than about 1000 g/mol, less than about 800 g/mol, or less than about 500 g/mol. In further preferred embodiments, the active agents are one or more small molecule drugs that bind to melanin. In some embodiments, one or more complexation agents are added to enhance the stability of the crystals or microcrystals of the small molecule drugs. Exemplary complexation agents include one or more lipids and nucleic acids.

Crystallization is widely used as a powerful purification tool and as a demonstration of chemical purity, as the process of crystallization purifies active agents.

Crystals of active agents can be formed under conditions which effect precipitation in a solution having an optimal pH, optimal concentration of each precipitant and optimal presence of salts/additives. In some embodiments, crystallization of active agents is carried out using existing “Seed” crystals, to initiate growth of new crystals.

Typically, the size of microcrystals is predominantly between about 0.1 microns to 1,000 microns, between about 1 micron to 500 microns, or preferably between about 5 microns to 250 microns. Dimensions of microcrystals are typically determined by light microscopy. If crystals of active agents are produced larger than about 0.1 microns to 1,000 microns, the crystals can be broken into smaller fragments amenable for administration as microcrystals. Typically, the microcrystals have a size suitable for administration by 25-, 27- or 30-gauge needles. In preferred embodiments, the size of microcrystals is less than 500 microns, less than 400 microns, less than 300 microns, less than 200 microns or less than 100 microns.

The microcrystal formulations typically include active agent(s) in crystalline form at a concentration of about 0.1 mg/ml excipient or suspending agent to about 1,000 mg/ml excipient or suspending agent.

In preferred embodiments, ion complexation is preferred to form microcrystals. Ion complexation generally refers to as a process where two water soluble compounds with opposing charge are mixed together to form an insoluble complex. In some embodiments, ion complexation for formulating microcrystals results in about 20-80% of an active agent, 30-70% of an active agent, 40-60% of an active agent, or about 50% of an active agent in the microcrystals. In particular embodiments, microcrystals contain about 50% loading of an inhibitor of dual leucine zipper kinase (DLK).

The microcrystals of active agents can be either crystalline, amorphous, or a mixture thereof. It is generally preferred that the drug be present in the formulation in crystalline form.

In preferred embodiments, compositions and methods useful for in vivo delivery of one or more active agents, preferably melanin-binding active agents, are in the form of microcrystals that are suitable for parenteral administration in aqueous particulate suspension. In further preferred embodiments, the formulations including microcrystals free of non-crystalline aggregates. The microcrystal suspension is typically suited for administration by 25-, 27- or 30-gauge needles.

In particular embodiments, the microcrystals are prepared by complexing sunitinib malate with pamoic acid disodium through ionic interaction followed by crystallization to generate microcrystals. Typically, the microcrystals formed with sunitinib malate and pamoic acid have a mean particle size of 25±7 μm and width of 10±2 μm (s.d.). Typically, the microcrystals tend to have a rod-like morphology.

Methods of crystallization are discussed in more detail below.

### C. Pharmaceutical Formulations

Pharmaceutical compositions including microcrystals of one or more active agents such as sunitinib and pamoic acid may be formulated in a conventional manner using one or more physiologically acceptable carriers including excipients and auxiliaries which facilitate processing of the microcrystals into preparations which can be used pharmaceutically.

Formulation is dependent upon the route of administration chosen. In preferred embodiments, the compositions are formulated for parenteral delivery. In some embodiments, the compositions are formulated for intraocular injection. Typically, the compositions will be formulated in an isotonic solution to the eye, about 300 mOsm/L, for example, in sterile saline or buffered solution for injection into the tissues or cells to be treated in the eye. The compositions can be stored frozen or lyophilized in single use vials for rehydration immediately before use. Other means for rehydration and administration are known to those skilled in the art.

Pharmaceutical formulations contain one or more microcrystals of active agents for treating, preventing or diagnosing eye diseases or disorders in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably materials which are generally recognized as safe (GRAS), and may be administered to an individual without causing undesirable biological side effects or unwanted interactions. The pharmaceutical excipients should not reduce the stability of the microcrystals.

Generally, pharmaceutically acceptable salts can be prepared by reaction of the free acid or base forms of an agent with a stoichiometric amount of the appropriate base or acid in water or in an organic solvent, or in a mixture of the two. Generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, M D, 2000, p. 704. Examples of ophthalmic drugs sometimes administered in the form of a pharmaceutically acceptable salt include timolol maleate, brimonidine tartrate, and sodium diclofenac.

The compositions of microcrystals are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. An animal model can also be used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions of microcrystals are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions of microcrystals formulated for administration by parenteral (intraocular, intravitreal, intramuscular, intraperitoneal, intravenous (IV) or subcutaneous injection) and enteral routes of administration are described.

### III. Methods of Making Crystalline Compositions

Methods for preparing pharmaceutical formulations of microcrystals typically include one or more steps of crystallizing an active agent. Active agents can be crystallized according to methods known in the art for the crystallization of molecules and macromolecules. Typically, crystallization involves purifying and concentrating an active agent to a concentration between 0.1 and 1,000 mg/ml in a suitable solvent for crystallization, and incubating the solution under conditions suitable for crystallization. In some embodiments, the methods include a step for purifying and/or isolating the active agent. Methods of purifying and identifying one or more molecules from a mixture of molecules are known in the art. In some embodiments, the step of purifying an active agent includes solubilizing the agent in a desired solvent, at a desired concentration.

Crystallization conditions can vary between different active agents. Therefore, in some embodiments, the methods include one or more steps of determining crystallization conditions for one or more active agents. Methods and assays for screening of crystallization conditions can be carried out according to assays and methods known in the art. In some embodiments, the presence of factors including concentration of the agent, crystallization technique, solvent, pH, heat, light and other atmospheric conditions are altered to cause changes in one or more of the rate of crystallization, the size, shape and stability of crystals.

In some embodiments, the methods produce crystals of a single active agent. In other embodiments, the methods crystallize two or more active agents together within the same crystal. In some embodiments, the methods produce crystals of two or more different active agents together in a complex. Typically, crystals of two or more active agents within a complex are formed following admixture of the two or more agents together and crystallization of the resulting complex from within the mixture. Therefore, in some embodiments, the methods include one or more steps of mixing together two or more active agents in a solution under conditions suitable to form a complex between the two or more active agents within a solution, and subsequent crystallization of the soluble complex. In an exemplary embodiment, crystals of Sunitinib in complex with Pamoic acid are prepared by complexing sunitinib malate with pamoic acid disodium through ionic interaction, followed by crystallization to generate microcrystals.

Typically, the methods include one or more steps to isolate the microcrystals from the solution of reagents following crystallization, and/or to place crystals into a desired solvent. Methods for purifying crystals are known in the art and can include one or more of centrifugation, filtration, dialysis, solvent washing and chromatography. In some embodiments, the methods include one or more steps for assessing the content of microcrystals. For example, the methods can identify the size of crystals, and/or the concentration of the active agent(s) within the crystals. Therefore, in some embodiments, the methods include one or more steps to process crystals of active agents, for example, one or more steps to select or fractionate crystals, or to remove crystals, or to break crystals into smaller pieces, for example, fragments having dimensions of between about 0.1 μm and 1,000 μm in size.

In an exemplary method, microcrystals of a complex including sunitinib and pamoic acid are prepared. The methods first form a complex between 60 mg of sunitinib maleate and 600 mg of pamoic acid disodium, each dissolved in 15 mL of distilled water in separate containers. The methods include mixing the solutions of active agents together and incubating for 2 h at room temperature for ionic complexation. Following complexation, the methods separate the sunitinib and pamoic acid complexes from the free sunitinib malate and pamoic acid disodium by centrifuging at 10,000 rpm for 30 min. The supernatant is removed and washed with 15 mL of distilled water 3 times. The methods then include crystallizing the sunitinib and pamoic acid complexes. For crystallization, 2.5 ml of the 75% ethanol in water is added to the complex and mixed until fully dissolved. The solution is then stirred at 60 rpm for 2 h to facilitate crystallization. The methods collect crystals by filtering through a 20 μm Nylon Net filter to remove larger crystals followed by washing with 20 mL of ultra-pure distilled water with a 35 μm cell strainer. The methods identify the size and shape of crystals by light microscopy and process the crystals as necessary to obtain the required size and amount of crystals.

In other embodiments, the methods include crystallizing a first active agent in a solution containing only a first active agent and adding a second or further active agent to the crystalline form of the first active agent. For example, in some embodiments the methods include one or more steps of contacting a crystalline form of a first active agent with a solution or powder containing one or more additional active agents under conditions suitable for the crystalline form of the first active agent to absorb or otherwise complex with the second or further active agent(s) in the solution or powder.

The methods typically include one or more steps to formulate microcrystals in an excipient that is pharmaceutically acceptable for administration into the eye. An exemplary excipient is saline. Typically, the amount of the active agent(s) in the formulation is adjusted according to the nature of the active agent, the intended use, and the needs of the recipient. In some embodiments, the weight and amount of crystals includes within the formulation are used to determine the dosage of the active agent. In preferred embodiments, the volume of the formulation for administration is between 0.1 μl and 1,000 μl.

### IV. Methods of Use

Delivery of active agents to the eye in crystalline form can treat and prevent diseases and disorders of the eye over a sustained period following a single administration. Typically, administration of the active agent in crystalline form provides enhanced therapeutic benefit to the subject as compared with the same amount of the active agent administered to the subject in non-crystalline form. The steady dissociation of crystals into molecules of active agents within the eye provides a sustained release of active agents within the confined compartment of the eye, providing long-lasting therapeutic efficacy following a single administration. This is further enhanced in the case where the agent binds to a ligand or receptor within the eye, such as sunitinib binding to melanin.

Methods of using microcrystal compositions for treatment of eye diseases and disorders are described. Methods of delivering active agents by injection of microcrystals into the eye are also provided. In some embodiments, the microcrystal compositions are administered by injection into the posterior compartment of the eye. Methods for delivery, accumulation, and intraocular release of one or more active agents for the treatment, prevention and diagnosis of eye diseases or disorders are described.

In some embodiments, formulations of microcrystals in a pharmaceutically acceptable excipient are administered locally into the eye of a subject having an eye disease or disorder, or at risk of having an eye disease or disorder. In a preferred embodiment, the microcrystals of active agents are delivered directly into one or more compartments of the eye by injection. Typically, the microcrystals are injected as a suspension in solution, in a volume of between about 0.1 μl and about 1,000 μl, preferably between about 1 μl and 50 μl. In a particular embodiment, the methods deliver microcrystals of Sunitinib malate conjugated to pamoic acid by intravitreal, sub-conjunctival, or supra-choroidal injection into the eye for treatment and prevention of an eye disease, such as glaucoma.

In some examples, the compositions of microcrystals retain an effective concentration of one or more active agents at or near the site of administration and/or the target site for an extended period of time, for example, more than 1 day, more than 2 days, more than 3 days, more than 4 days, more than 5 days, more than 6 days, or more than a week. In preferred cases, the compositions of microcrystals are administered once a week or less frequently, once every other week or less frequently, once every month or less frequently, once every other month or less frequently. In some embodiments, administering compositions of microcrystals increases the concentration of one or more active agents at or near the site of application by 2-fold, 3-fold, 4-fold, 5-fold, 6-fold, 7-fold, 8-fold, 9-fold, 10-fold, or more than 10-fold compared to active agents delivered without microcrystals, for example, in saline solution. When the compositions of microcrystals are applied to the eye, the mucosal sites with increased concentration of active agents include one or more of cornea, aqueous humor, sclera, conjunctiva, iris, lens, retina, and retinal pigment epithelium.

In some examples, the methods deliver microcrystals of active agents (e.g., sunitinib malate) to the retina and/or choroid in an amount effective to reduce retinal and/or choroidal neovascularization by 10%, 20%, 30%, 40%, 50%, or more than 50% compared to active agents delivered without microcrystals, for example, in saline solution.

In other examples, the methods deliver microcrystals of active neuroprotective agents (e.g., sunitinib malate) to the retina in an amount effective to increase the survival of retinal ganglion cells following optic nerve injury, and/or to increase the expression of γ-synuclein and/or βIII tubulin in retinal ganglion cells following optic nerve injury by 2-fold, 3-fold, 4-fold, 5-fold, or more than 5-fold compared to active agents delivered without microcrystals, for example, in saline solution.

In further examples, the methods deliver microcrystals of active agents (e.g., brinzolamide, sunitinib) to the eye in an amount effective to lower intraocular pressure (IOP) by 10%, 20%, 30%, 40%, 50%, or more than 50% compared to those delivered without microcrystals, for example, in saline solution within less than 2 hours, 4 hours, 6 hours, 8 hours, 10 hours, 12 hours, or 24 hours.

In other examples, the methods deliver microcrystals of active agents (e.g., sunitinib) to the eye in an amount effective to increase tear production by 10%, 20%, 30%, 40%, 50%, or more than 50% compared to those delivered without microcrystals, for example, in saline solution within less than 2 hours, 4 hours, 6 hours, 8 hours, 10 hours, 12 hours, or 24 hours.

### A. Methods of Treating Eye Disorders and Diseases

The compositions are suitable for treating one or more diseases, conditions, and injuries in the eye. The compositions and methods are also suitable for prophylactic use. In preferred embodiments, the compositions of microcrystals are administered in an amount effective to treat one or more diseases, conditions, and injuries in the eye in the subject in need thereof without any associated toxicity. In some embodiments, administration of a single dose of microcrystals into the eye provides sustained release of the active agents within the eye for a period of one, two, three, four, five, six, seven, eight, nine or ten, or twenty days or weeks or months following administration. Therefore, in some embodiments, administration of a single dose of microcrystals into the eye treats or prevents one or more diseases, conditions, or injuries of the eye for a period of one, two, three, four, five, six, seven, eight, nine or ten, or twenty days or weeks or months following administration. In other embodiments, administration of a single dose of microcrystals into the eye reduces or prevents one or more symptoms of eye disease, or a condition or injury of the eye for a period of one, two, three, four, five, six, seven, eight, nine or ten, or twenty days or weeks or months following administration. Typically, the amount of the active agent administered in crystalline form effective to treat or prevent one or more diseases, conditions, or injuries of the eye is less than the amount of the same active agent effective to treat or prevent one or more diseases, conditions, or injuries of the eye in non-crystalline form.

In some cases, the subject to be treated is a human. In some cases, the subject to be treated is a child, or an infant. All the methods can include the step of identifying and selecting a subject in need of treatment, or a subject who would benefit from administration with the described compositions.

i. Ocular Disorders and Diseases to be Treated

The compositions and methods are suitable for treating and preventing one or more diseases, disorders, and conditions associated with the eye. The compositions and methods are particularly suited for treating and alleviating one or more symptoms associated with glaucoma. In some embodiments, the disclosed compositions or formulations thereof are used to alleviate pain, facilitate healing, and/or reduce or inhibit scarring.

Examples of eye disorders that may be treated according to the disclosed compositions and methods include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatrical scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age-related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. Other disorders including injury, burn, or abrasion of the cornea, cataracts and age related degeneration of the eye or vision associated therewith.

In some cases, the eye disorder to be treated are retinal diseases. Retinal diseases can affect any part of the retina, a thin layer of tissue on the inside back wall of the eye. Exemplary retinal diseases include retinal tear, retinal detachment, diabetic retinopathy, epiretinal membrane, macular hole, macular degeneration, and retinitis pigmentosa. In preferred case, the eye disorder is age-related macular degeneration (AMD). Age-related macular degeneration (AMD) is a neurodegenerative, neuroinflammatory disease of the macula, which is responsible for central vision loss. The pathogenesis of age-related macular degeneration involves chronic neuroinflammation in the choroid (a blood vessel layer under the retina), the retinal pigment epithelium (RPE), a cell layer under the neurosensory retina, Bruch's membrane and the neurosensory retina, itself.

The formulations may be administered to animals, especially mammalian animals for treating or alleviating pain, disease, disorder, infection, or injury of the eye.

### B. Dosage and Effective Amounts

Dosage and dosing regimens are dependent on the severity and location of the disorder or injury and/or methods of administration, and can be determined by those skilled in the art. A therapeutically effective amount of the microcrystal composition used in the treatment of eye disorders and/or diseases is typically sufficient to reduce or alleviate one or more symptoms of the eye disorders and/or diseases in the subject. Exemplary symptoms include eye pain or eye discomfort, such as soreness, dryness, burning, stinging or aching sensations, reduced vision loss or visual acuity, reduced ability to read or interpret colors, reduced movement of the eye, or one or more physiological parameters, such as expression of γ-synuclein and/or 111 tubulin in retinal ganglion cells, increased intra-ocular pressure, vascular leakage from retinal blood vessels, and retinal and/or choroidal neovascularization.

Preferably, the active agents do not target or otherwise modulate the activity or quantity of healthy cells not within or associated with the diseased/damaged tissue, or do so at a reduced level compared to cells associated with the diseased/damaged eye tissue. In this way, by-products and other side effects associated with the compositions are reduced.

Therefore, administration of the microcrystal compositions leads to an improvement, or enhancement, function in an individual with an ocular disease, injury, or disorder.

The actual effective amounts of the microcrystal compositions can vary according to factors including the specific agent administered, the particular composition formulated, and the age, weight, condition of the subject being treated, as well as the disease or disorder. Dosage can vary, and can be administered in one or more dose administrations weekly, monthly, bimonthly, once every six month or less frequently. Guidance can be found in the literature for appropriate dosages for given classes of pharmaceutical products. Optimal dosing schedules can be calculated from measurements of drug accumulation in the body and/or eye of the subject or patient. Persons of ordinary skill in the art can easily determine optimum dosages, dosing methodologies and repetition rates. Optimum dosages can vary depending on the relative potency of individual pharmaceutical compositions and can generally be estimated based on EC₅₀s found to be effective in in vitro and in vivo animal models.

Dosage forms of the pharmaceutical composition including microcrystals of active agents are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose. In some embodiments, the dosage unit suitable for use are between 5 μg/dosage unit and about 100 mg/dosage unit, inclusive; between about 10 μg/dosage unit and about 20 mg/dosage unit, inclusive; and between about 100 μg/dosage unit and about 10 mg/dosage unit, inclusive; and between about 200 μg/dosage unit and about 5 mg/dosage unit, inclusive; and between about 500 μg/dosage unit and about 3 mg/dosage unit, inclusive.

In general, the timing and frequency of administration will be adjusted to balance the efficacy of a given treatment or diagnostic schedule with the side effects of the given delivery system. Exemplary dosing frequencies include continuous infusion, single and multiple administrations such as weekly, monthly or yearly dosing.

In some embodiments, dosages are administered weekly, every two weeks or less frequently in an amount to provide a therapeutically effective increase in the target tissue of the therapeutic agent. In preferred cases, the compositions are formulated for controlled release such as in a microcrystal suspension, wherein the composition is administered as a single dose that is repeated on a regimen of once a week, or less frequently.

It will be understood by those of ordinary skill that a dosing regimen can be any length of time sufficient to treat the disease or disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### C. Combination Therapies and Procedures

The microcrystal compositions can be administered alone or in combination with one or more conventional therapies. In some embodiments, the conventional therapy includes administration of one or more microcrystal compositions in combination with one or more additional active agents. The combination therapies can include administration of the active agents together in the same admixture, or in separate admixtures. Therefore, in some embodiments, the pharmaceutical composition includes microcrystals and one or more additional active agent. The one or more additional active agent can be administered in the same pharmaceutical formulation, or in a separate formulation delivered at the same time, or at a different time as the microcrystals. In some embodiments, the treatment regimen includes multiple administrations of microcrystals to the same eye, or to different eyes. Such formulations typically include an effective amount of an agent targeting the site of treatment. The additional active agent(s) can have the same or different mechanisms of action. In some embodiments, the combination results in an additive effect on the treatment of the eye disease or condition. In some embodiments, the combinations result in a more than additive effect on the treatment of the disease or disorder.

The additional therapy or procedure can be simultaneous or sequential with the administration of the microcrystal composition. In some embodiments, the additional therapy is performed between drug cycles or during a drug holiday that is part of the composition's dosage regime. Therefore, in some embodiments, a formulation of microcrystals is administered in a single dose to a subject 1, 2, 3, 4, 5, 6, 7, 8, 9 or 10 minutes, hours or days before or after administration of an additional active agent to the same subject for the treatment of the same eye disease or condition.

In some embodiments, the compositions and methods are used prior to or in conjunction, subsequent to, or in alternation with treatment with one or more additional therapies or procedures.

### D. Controls

The therapeutic effect of microcrystal compositions including one or more active agents can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the microcrystal composition on a particular symptom, pharmacologic, or physiologic indicator can be compared to an untreated subject, or the condition of the subject prior to treatment, or to the same amount of active agent in non-crystalline from. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the microcrystal treatment is compared to a conventional treatment that is known the art. In some embodiments, an untreated control subject suffers from the same eye disease or condition as the treated subject.

### V. Kits

The compositions can be packaged in kit. The kit can include a single dose or a plurality of doses of a composition including one or more active agents and instructions for administering the compositions Specifically, the instructions direct that an effective amount of the composition be administered to an individual with a particular eye disease/disorder as indicated. The composition can be formulated as described above with reference to a particular treatment method and can be packaged in any convenient manner. These may be unit dosage forms such as a pre-loaded syringe and one or more needles with gauge suitable for administration to the eye, for example, 25-, 27- or 30-gauge needles.

The present invention will be further understood by reference to the following non-limiting examples.

### Examples

### Example 1: Microcrystal Formulation of Sunitinib

### Methods

### Material Sources

Sunitinib malate was purchased from LC Laboratories. Pamoic acid disodium was purchased from Tokyo Chemical Industry. N-desethyl sunitinib and Sunitinib-d10 were purchased from Toronto Research Chemical. Phosphate buffered saline (PBS), Neurobasal media, calcein AM (acetomethoxy derivate of calcein), ethidium homodimer, Hoechst 33342, B-27, N-2, L-glutamine, penicillin/streptomycin, goat anti-Rabbit IgG (H+L) Alexa Fluor 568, 4′,6-diamidino-2-phenylindole dihydrochloride (DAPI), 2-mercaptoethanol, bovine serum albumin and Fluoromount-G were purchased from Thermo Fisher Scientific. CellTiter-Glo luminescent cell viability assay was purchased from Promega. Goat anti-mouse IgG H&L Alexa Fluor® 647 was purchased from Abcam. High-performance liquid chromatography (HPLC) grade acetonitrile and water were purchased from Fisher Scientific. Nylon net filters (20 μm) were purchased from Millipore. Mesh strainers (35 μm) were purchased from Corning. Deuterated dimethyl sulfoxide D-6 was purchased from Cambridge Isotope Laboratories. Polysorbate 80, dextran 40, PS0, PS68, L61 Lutrol F127, trifluoroacetic acid, Triton X-100, diethyl ether, acetone, and biological grade ethanol were purchased from Sigma Aldrich. Methylcellulose (400 cP) was purchased from Spectrum Chemical. Reverse-action forceps were purchased from World Precision Instruments. Rabbit-anti γ-Synuclein was purchased from GeneTex. Mouse-anti β-III tubulin was purchased from BioLegend. Nylon sutures (10-0) were purchased from Ethicon. Neomycin, polymyxin B, and bacitracin zinc ophthalmic ointment was purchased from Akorn. ISOTON® Diluent was purchased from Beckman Coulter.

### Sunitinib-Pamoate Complex (SPC) Synthesis and Characterization

Negatively charged hydrophobic salts, including pamoic acid disodium salt, linoleic acid sodium salt, sodium stearate, and sodium decanoate, were screened for complexation with sunitinib malate. The hydrophobic salts and sunitinib malate were each dissolved separately at 0.5 mg/mL in 15 mL of ultra-pure, endotoxin-free water. Linoleic acid sodium salt, sodium stearate, and sodium decanoate formed an oily, cloudy solution when mixed 1:1 with sunitinib malate. In contrast, pamoic acid disodium formed a solid precipitate when mixed with sunitinib malate, and thus, was used for further formulation. Various weight ratios (1:1, 2:1, 5:1, 8:1, 10:1, 20:1) of pamoic acid disodium salt to sunitinib and different temperature settings (freezer, refrigerator, room temperature, and water bath at 37 and 54° C.) were screened for complexation efficiency after incubation for 24 h by visually observing the size of the pellet after centrifugation at 4000 rcf for 30 min. It was found that a minimum of 1 h complexation time was required to maximize yield and room temperature was suitable (not shown). To make the sunitinib-pamoate complex (SPC) for particle formulation, sunitinib malate (60 mg) and pamoic acid disodium salt (600 mg) were dissolved separately in 15 mL of ultra-pure, endotoxin-free water and were further mixed and by brief vortexing at room temperature for 5 s. The mixture was kept in the dark for 2 h, then collected by centrifugation at 4000 rcf for 30 min, and lyophilized (Labconco) for further characterization. SPC drug loading study was determined by measuring the sunitinib concentration from the SPC dissolved with DMSO. The SPC were weighed and dissolved with 1 mL of the DMSO and further diluted 200× with the DMSO. The sunitinib concentration were measured at λₑₘ=540 and further used for calculating the drug loading percentage based on the original weighed SPC (n=6). Nuclear magnetic resonance (NMR) was also used to determine the ratio of sunitinib to pamoate in SPC. Sunitinib malate, pamoic acid, and SPC were separately dissolved in deuterated dimethyl sulfoxide. ¹H NMR spectra were recorded on a Bruker spectrometer (500 MHz), and the data were processed using iNMR software. ¹H chemical shifts were reported in ppm (δ) and the DMSO peak was used as an internal standard. The molar ratio of sunitinib malate and pamoic acid disodium in the SPC complex was determined by the taking the ratio of sunitinib peak at δ=1.2 to pamoic acid peak 6=4.8. To measure solubility, 5 mg of sunitinib malate or SPC complex was placed in microcentrifuge tubes with 1 mL of PBS. The samples were then placed on an orbital shaker (150 rpm) in an incubator at 37° C. After 7 days, samples were collected and centrifuged at 17,000 rcf for 30 min. The supernatant was collected, and concentrations were measured using HPLC (Shimadzu Prominence LC-20). Supernatant samples were mixed 1:10 with acetonitrile containing 0.1% trifluoroacetic acid. Acetonitrile and water was used as a mobile phase at a ratio of 55:45. Samples were eluted isocratically at a flow rate of 1 mL/min through a C18-reversed phase column at 40° C. UV absorbance was monitored at 431 nm.

### SPC Microcrystal Formulation and Characterization

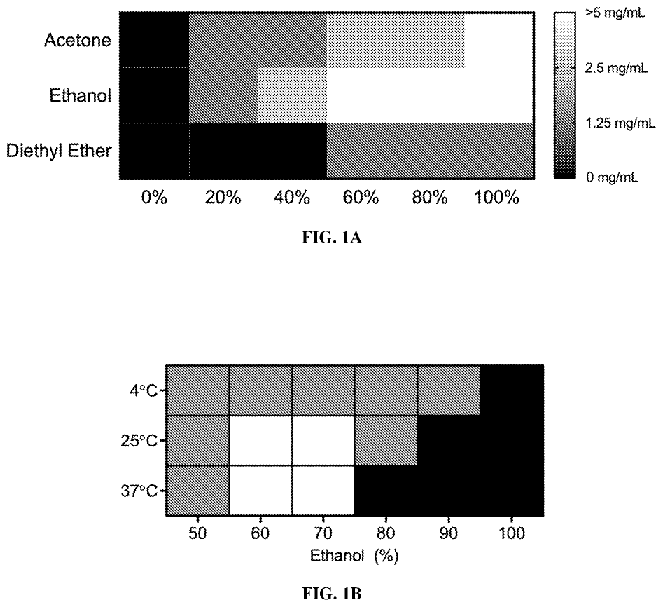

Different volume ratios (0%, 20%, 40%, 60%, 80%, 100%) of acetone, ethanol, and diethyl ether in water were screened for dissolving SPC for recrystallization, where the SPC was most soluble in ethanol/water (FIG. 1A). The solutions of SPC in ethanol:water (60% to 100%) were then stirred at different temperatures (refrigerator, room temperature, water bath at 37° C.) to evaporate solvent and facilitate crystallization. It was found that microcrystals formed when using 60-70% ethanol as the solvent, whereas other mixtures either remained solutions or formed uncontrolled aggregates (FIG. 1B). To make the final SPC microcrystal formulation, SPC solids (78 mg±5.2 mg) were dissolved by adding 2.7 mL of 70% ethanol/water and vortexing vigorously for 5 min. The solution was then stirred in a fume hood at 60 rpm for 2 h to evaporate solvent and facilitate crystallization. The resulting SPC microcrystals were then collected by pouring through a 20 μm net filter, followed by three times of washing each with 50%, 20%, and 0% of ethanol in water. Various surface stabilizers were then added to improve crystal stability. Suspending the crystals in polysorbate 80 (0.01%-1%), Pluronic F68 (0.05%-1%), Pluronic F127 (0.005%-1%), and Pluronic L61 (0.05%-0.1%) all resulted in either an increase in the heterogeneity of the crystal size or crystal dissolution within 24 h after addition (confirmed by visual observation under a light microscope). In contrast, the size of the microcrystals was stable at room temperature without a stabilizer when kept at a concentration of at least 5 mg/mL, and thus a stabilizer was not incorporated in the final formulation. Drug loading was calculated by dissolving a known mass of lyophlilized crystals and determining the sunitinib content by HPLC (details above). The drug loading as a percentage of the total mass was found to be 50.5±2.88% (n=6). The final yield was 37±7.2% (n=3) with respect to the starting amount of sunitinib malate. Crystal size and morphology was characterized both by Coulter counter (Multisizer 4e, Beckman Coulter Life Sciences) and light microscopy. To reduce microcrystal aggregation observed when adding to the Coulter ISOTON® Diluent, 1% (w/w) F127 was added immediately prior to size measurements. SPC microcrystals (200 μL at 5 mg/mL sunitinib in 1% F127) were added to 100 mL of ISOTON diluent. The particle size was quantified until the total particle count reached 100,000 particles. To visualize microcrystal morphology, 5 μL of solution (40 mg/mL in 1% (w/w) F127 to prevent aggregation upon drying) was placed on a glass slide with a coverslip and imaged with a bright field microscope (Nikon ECLIPSE Ni) at 40×. At least 3 images were taken, and n=10 crystals measured per sample (n=3 samples). ImageJ software was used to measure the size of the SPC crystals (25.7±6.6 μm by 10±2 μm). For the crystal stability test, 200 μL of 5 mg/mL of SPC microcrystals in water were aliquoted into 1.5 mL Eppendorf tubes and kept at room temperature in the dark for up to 180 days. The crystal size was measured at 0, 21, 35, 120, and 180 days by Coulter counter. A powder x-ray diffractometer (XRD, X'Pert PRO MPD) was used to characterize SPC microcrystal crystallinity. SPC microcrystals suspended in water were lyophilized (Labconco) for 1 week to obtain powdered SPC. The SPC powders were packed to the sample holder with a flat surface and located against the reference plane in the center of the goniometer. The start and end angles were set to 5° and 100°, with a step size of 0.020 and time per step set as 10 s. The tension was set to 45 kV and the current to 40 mA. Background subtraction and smoothing were applied to the dataset for qualitative analysis. The intensity of the x-ray diffraction patterns was recorded as a function of the angle of the detector (20).

### Results

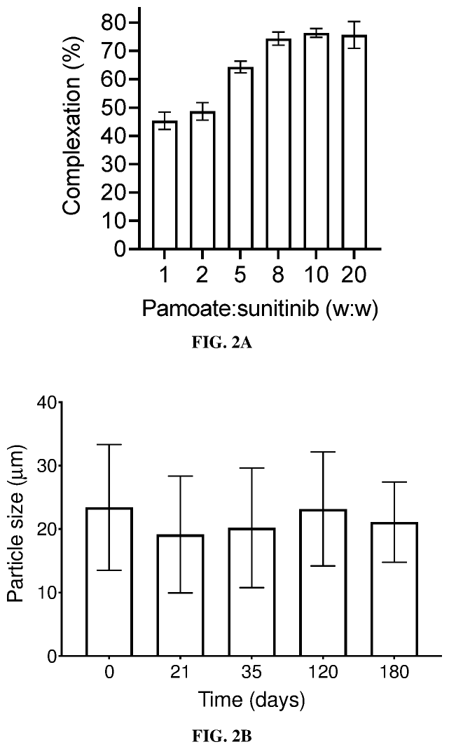

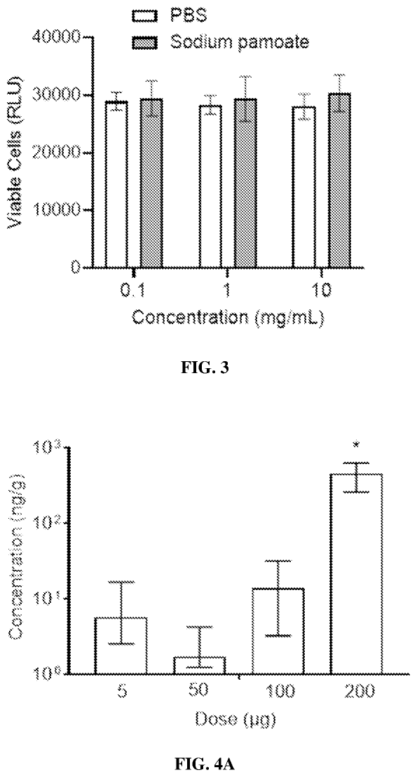

Hydrophobic anionic salts were first screened as potential ion pairs for sunitinib malate and identified pamoic acid disodium salt as suitable for producing an insoluble complex. As shown in FIG. 2A, the complexation efficiency increased with increasing excess of pamoic acid, with a plateau around 8-10 fold excess of pamoic acid resulting in complexation of ˜75% of the sunitinib malate in solution. Using NMR, it was confirmed that the ratio of sunitinib to pamoate in the sunitinib-pamoate complex (SPC) was 1:1. This was consistent with the dicarboxylic acid structure of pamoic acid, which is similar to the conventional malic acid used in the water-soluble pharmaceutical salt. However, the water solubility of the SPC was dramatically decreased ˜34-fold compared to sunitinib malate, 15.3±1.4 μg/mL vs. 525±63 μg/mL, respectively. Next assays were carried out to ensure that pamoic acid was not cytotoxic to RGCs. Pamoic acid was found to have no detrimental effect on primary mouse RGC survival compared to PBS at concentrations as high as 10 mg/mL (FIG. 3). The SPC was then dissolved in 70% ethanol/water and recrystallized to form micron sized particles, which were confirmed to by crystalline by x-ray diffraction. Surprisingly, the SPC microcrystals showed relatively stable size as measured by the Coulter counter without additional surface stabilizers when stored at or above 5 mg/mL at room temperature for up to 180 days (23.4±9.9 μm on day 0 vs. 21.1±6.3 μm at 180 days). Though the SPC microcrystals showed rod-shaped morphology, which when measured by image analysis showed approximate length of 25±7 μm and width of 10±2 μm. Volume mean size (μm) of SPC microcrystals over a period of 180 days at room temperature is shown in FIG. 2B.

### Example 2: Ocular Pharmacokinetics and In Vivo Therapeutic Efficacy of SPC Microcrystals

### Methods

### Retinal Ganglion Cell (RGC) Viability Assay

Mouse RGCs were purified as described ny Welsbie, D. S., et al., Proceedings of the National Academy of Sciences of the United States of America, 2013. 110(10): p. 4045-4050. Purified cells were plated on a poly-D-lysine/laminin coated 384-well culture dish at 4,000 cells per well and cultured in Neurobasal media supplemented with B-27, N-2, L-glutamine, and penicillin/streptomycin. Sunitinib malate (1 μM) was dissolved in dimethyl sulfoxide (DMSO) and added at the same time cells were plated (250 nL into 50 μL of media) to promote RGC survival. Then, either PBS or different volumes of sodium pamoate dissolved in DMSO was added at the concentrations indicated (0.1, 1, 10 mg/mL) to determine whether there was any effect on RGC survival. A PBS control was included for each pamoic acid concentration to account for dilutional effects. After cells were cultured for 72 h at 37° C., viability was measured by CellTiter-Glo (Promega) luminescence.

### Animal Welfare Statement

All experimental protocols were approved by the Johns Hopkins Animal Use and Care Committee. All animals were handled and treated in accordance with the Association for Research in Vision and Ophthalmology Statement for Use of Animals in Ophthalmic and Vision Research. For rats and rabbits, sex was not specified when ordering any species, and approximately equal amounts of male and female animals were used as provided. For the pigs, n=2 male and n=2 female animals were used. Brown Norway and Wistar rats, and New Zealand White rabbits were obtained from Charles River. Yorkshire pigs were obtained from Archer Farm. All animals received topical anesthesia (0.5% proparacaine hydrochloride) prior to ocular injections and procedures. Animals were anesthetized prior to euthanasia.

### Pharmacokinetics

### Effect of Injection Volume and Dose on Drug Concentration in the Posterior Segment.

Wistar rats (6-8 weeks old) and New Zealand White rabbits (2-3 kg) were used. Animals received a unilateral subconjunctival injection of SPC microcrystals using a 27-gauge needle. To look at the effect of dose in rats, four different doses of SPC microcrystals (5, 50, 100, 200 μg) were suspended in carrier fluid containing 0.04% (w/v) PS80 with 0.5% (w/v) methylcellulose in saline and injected in 5 μL volume. At 7 days after the injection, the eyes were enucleated and separated into the retina and choroid/retinal pigmented epithelium (Ch/RPE) for analysis of sunitinib and N-desethyl sunitinib content. To look at the effect of injection volume, SPC microcrystals were suspended in carrier fluid containing 0.04% (w/v) PS80 with 0.5% (w/v) methylcellulose in saline for the 5 μL injections. For the 100 μL (rats) and 500 μL (rabbits) injections, the overall material amounts (200 μg sunitinib, 2 μg PS80, 25 μg methylcellulose) in saline were matched. At 7 days after the injection, the eyes were enucleated and separated into the retina and choroid/retinal pigmented epithelium (Ch/RPE) for analysis of sunitinib and N-desethyl sunitinib content. As sunitinib and N-desethyl sunitinib showed similar potency in the RGC survival experiments in vitro, drug levels were combined and reported.

### Drug Concentrations in the Posterior Segment.

Brown Norway rats (6-8 weeks old) and juvenile Yorkshire pigs (20-30 kg) were used. Rats received a 5 μL unilateral subconjunctival injection of SPC microcrystals (200 μg sunitinib, 0.04% PS80, 0.5% methylcellulose) using a 27-gauge needle. At 1, 3, 5, and 20 weeks after the injection, the eyes were enucleated and separated into the retina and Ch/RPE for analysis of sunitinib and N-desethyl sunitinib content. Drug levels were combined and reported. Pigs received a 5 μL (200 μg sunitinib, 0.04% PS80, 0.5% methylcellulose) or 50 μL (2 mg sunitinib, 0.04% PS80, 0.5% methylcellulose) unilateral subconjunctival injection of SPC microcrystals using a 27-gauge needle. After 7 days, the animals were sacrificed, and the retina and Ch/RPE were collected for analysis for sunitinib malate and N-desethyl sunitinib content. Drug levels were combined and reported.

### Effect of Injection Volume on Drug Concentration in the Cornea.

Rats received a unilateral anterior subconjunctival injection of SPC microcrystals (200 μg sunitinib) using a 27-gauge needle. For the 5 μL injections, SPC microcrystals were suspended in carrier fluid containing 0.04% (w/v) PS80 with 0.5% (w/v) methylcellulose in saline. For the 100 μL injections in rats, the dose was matched (200 μg sunitinib, 2 μg PS80, 25 μg methylcellulose). At 7 days after the injection, the eyes were enucleated, and the cornea was excised for analysis of sunitinib and N-desethyl sunitinib content. As sunitinib and N-desethyl sunitinib have been shown to have similar potency in inhibiting VEGF (Numakura, K., et al., Oncotarget, 2018. 9(38): p. 25277-25284), drug levels were combined and reported.

### Measurement of Sunitinib and N-Desethyl Sunitinib in Ocular Tissues

All the obtained tissues were collected in pre-weighed tubes and stored in −80° C. freezer until analysis. Liquid chromatography-tandem mass spectrometry (LC-MS/MS) was used to measure drug concentrations as previously described (Kim, Y. C., et al., Nat Biomed Eng, 2020. 4(11): p. 1053-1062). Briefly, tissue samples were homogenized in 200-500 μL 1×PBS using Next Advance Bullet Blender before drug extraction. Sunitinib was extracted from 50 μL of tissue homogenates with 0.150 mL of acetonitrile containing 2.5 ng/mL of the internal standard, sunitinib-d10. After centrifugation, the top layer was then transferred autosampler vial for LC-MS/MS analysis. All ocular tissue samples were analyzed using a 1×PBS standard curve. The separation was achieved with a Waters Cortecs C18 (2.1×50 mm, 2.7 μm) column at room temperature using a gradient. Mobile phase A was water containing 0.1% formic acid and mobile phase B was acetonitrile containing 0.1% formic acid. The gradient started with mobile phase B held at 10% for 0.5 minutes and increased to 100% over 0.5 min; 100% mobile phase B was held for 1 min and then returned back to 10% mobile phase B and allowed to equilibrate for 1 min. The total run time was 3 min with a flow rate of 0.3 m/min. The column effluent was monitored using a Sciex triple quadrupole 5500 mass-spectrometric detector (Sciex, Foster City, CA, USA) using electrospray ionization operating in positive mode. The spectrometer was programmed to monitor the following MRM transition 399.1→283.2 for sunitinib, 371.2→283.2 for N-desethyl sunitinib and 409.1→283.2 for the internal standard, sunitinib-d10. A calibration curve for sunitinib was computed using the area ratio peak of the analysis to the internal standard by using a quadratic equation with a 1/x² weighting function over the range of 0.1-100 ng/mL for sunitinib with dilutions of up to 1:100 (v:v).

### Rat Optic Nerve Crush Model

Brown Norway rats (6-8 weeks old) were anesthetized and received unilateral subconjunctival injection of SPC microcrystals (200 μg/5 μL sunitinib, 0.04% PS80, 0.5% methylcellulose) using a 27-gauge needle. Two weeks before scheduled time points (3, 8, and 20 weeks), the rats were anesthetized and received topical anesthesia with 0.5% proparacaine hydrochloride. The temporal conjunctiva of the left eye was grasped with 0.12 mm toothed forceps and incised parallel to the limbus with sharp iris scissors. Dissection was performed using two pairs of curved blunt-tipped forceps, and the orbital fat and soft tissue were retracted to expose the orbital portion of the optic nerve. The optic nerve was crushed at a position 1.5-2 mm posterior to the globe using reverse-action forceps for 20 s. The orbital soft tissue was then repositioned over the nerve and the conjunctiva was left to close by secondary intention. After the procedure, topical bacitracin-neomycin-polymyxin ophthalmic ointment was applied to both eyes to prevent infection. Animals were sacrificed for tissue collection 14 days after the crush procedure.

### Rat Model of Laser-Induced Intraocular Pressure (IOP) Elevation

Translimbal laser treatment in rats induces a prolonged IOP increase and loss of RGCs judged by both axonal and cell body loss (Levkovitch-Verbin, H., et al., Invest Ophthalmol Vis Sci, 2002. 43(2): p. 402-10). Rats were given unilateral, subconjunctival injections of 200 μg SPC microcrystals (200 μg/5 μL sunitinib, 0.04% PS80, 0.5% methylcellulose) using a 27-gauge needle (day −7). Translimbal laser treatment was applied 7 days after microcrystal injection (day 0). Animals were sedated with a subcutaneous injection of ketamine-xylazine, and a drop of 0.5% proparacaine hydrochloride was used to anesthetize the eye. A 532-nm laser was used to induce ocular hypertension by scarring the trabecular meshwork as previously described. Each rat received a single treatment with 45 to 55 spots at 50-μm size, 0.6-W power, and 0.6-second duration by a surgeon who was masked to treatment group. Topical 5% erythromycin ointment was applied at the end of each procedure. IOP was measured on days 2, 5, 9, 14, 28, 35, and 40 following microcrystal injection using a TonoLab tonometer (iCare, Vantaa, Finland) calibrated for the rat eye under sedation by isoflurane inhalation. The tonometer was used according to the manufacturer's instructions with the magnetic probe in a horizontal position. Prior to measurements, sedation was induced for 3-4 minutes in an induction chamber. The rat was then transferred to a nose cone for IOP measurement. Three measurements, each consisting of the mean of 6 recordings, were taken of each eye. Topical anesthesia was not used for IOP measurement.

### Axial Length and Width Measurement

Measurement of axial length and width was described by Cone-Kimball, E., et al., Mol Vis, 2013. 19: p. 2023-39. Briefly, after sacrifice, intracardiac perfusion with 4% paraformaldehyde, and enucleation, IOP was set at 15 mm Hg with a needle placed in the eye and connected to a fluid-filled reservoir. Measurements were performed using a digital caliper (Instant-Read-out Precision Digital Caliper; Electron Microscopy Sciences, Hatfield, PA). The length was measured from the center of the cornea to a position just temporal to the optic nerve, and width was measured at the largest dimension at the equator, midway between the cornea and optic nerve.

### Optic Nerve Axon Counting

Rats were killed by exsanguination under deep ketamine-xylazine anesthesia. They were perfused transcardially with 4% paraformaldehyde in phosphate buffer, and the eyes with attached optic nerve were harvested. Eyes were fixed for an additional 1 hour in 4% paraformaldehyde prior to optic nerve dissection. Axonal loss was quantified using the method by Levkovitch-Verbin, H., et al., Invest Ophthalmol Vis Sci, 2002. 43(2): p. 402-10. Briefly, a cross section of the optic nerve was removed 1.5 mm posterior to the globe and postfixed in 1% osmium tetroxide in phosphate buffer. Nerves were processed into epoxy resin, sectioned at 1 μm, and stained with 1% toluidine blue. The area of the optic nerve cross section was measured by outlining its outer border at ×10 magnification on an image analysis system (Sensys digital camera and Metamorph software; Universal Imaging Corp., West Chester, PA). To measure the density and fiber diameter distributions, we captured images with a ×100 phase-contrast objective from 10 randomly spaced nerve regions. These were edited by a technician blinded to treatment group to eliminate nonneural objects, and the size of each axon internal to its myelin sheath (minimum diameter) and the density of axons per square millimeter were calculated for each image and for the entire nerve. The mean density was multiplied by nerve area to yield fiber number for each nerve. The total axon number in the glaucomatous eye was compared with pooled, control fellow eyes to yield a percentage loss value. The counting process was performed by observers masked to the protocol used in each nerve.

### Retinal Ganglion Cell Staining, Imaging, and Counting

For the crush studies, rats were sacrificed and the eyes harvested with the optic nerve attached. The retinas were removed, incised for flat mounting, and post-fixed for 1 h. For the laser studies, harvested eyes were post-fixed for 1 h (as described above), and the retinas were removed, incised, flat mounted, and fixed for an additional 24 h. Retinal tissue was then stored in phosphate buffer prior to staining. Retinas were then washed with 0.5% Triton-100 in PBS for 30 min, and incubated for 3 days at 4° C. in a solution containing rabbit-anti γ-synuclein (1:250 dilution), mouse-anti 111 tubulin (1:500 dilution), 1% Triton X-100, and 1% bovine serum albumin in PBS. The retinas were then washed three times with 0.5% Triton-100 in PBS, and further incubated overnight at 4° C. in a solution containing goat anti-mouse IgG H&L Alexa Fluor 647 (1:1000 dilution) and goat anti-rabbit IgG H&L Alexa Fluor 568 (1:1000 dilution) secondary antibodies in 1% Triton X-100, and 1% bovine serum albumin in PBS, or the same solution containing goat anti-rabbit IgG H&L secondary antibody Alexa Fluor 555 (1:1000 dilution), 1% Triton X-100, and 1% bovine serum albumin in PBS. The retinas were washed three times and incubated overnight in DAPI diluted 1:1000 in PBS. The stained retinal tissue was then mounted on a slide using Fluoromount-G. Several tissues from the laser group became too fragmented to image, leaving n=7 for the microcrystal treated group and n=12 for the sham group. For each retinal wholemount, 12 images were taken from the region 2-3 mm from the optic nerve (3 images per each of four retinal quadrants) at 40× magnification (Zeiss 710 Confocal Microscope). Confocal images were viewed in ImageJ (National Institute of Health) without modification of contrast or brightness. RGCs were identified by co-staining with DAPI and β-III tubulin. Investigators were masked as to the treatment when processing and imaging the tissues. RGCs were manually counted in a masked fashion by two independent observers. A third person was randomly assigned to count 9 out of the 12 images per retina flatmount to ensure consistency of counting results. If the count by the third observer was different than the average of the first two counters by more than ±10%, the images were recounted by all three observers before being averaged. For each animal, the number of RGCs was normalized to the healthy contralateral eye and reported as a percentage.

### Corneal Neovascularization Model

Sprague Dawley rats (6-8 weeks old) were used. Corneal neovascularization was induced by placing 10-0 nylon sutures superficially in the cornea, as previously reported with some modification (Kim, Y. C., et al., Investigative Ophthalmology & Visual Science, 2014. 55(11): p. 7376-7386). Rats were anesthetized and given a topical proparacaine solution as a local anesthetic of the ocular surface. One drop of iodine solution was instilled to disinfect the ocular surface. Two stitches of Nylon suture were placed in the right eye 1 mm apart in the superior cornea, 2-3 mm from the limbus, using an operating microscope. The placed suture remained in the cornea until the end of the experiment. Immediately after suture implantation, rats received a unilateral anterior 5 μL subconjunctival injection of SPC microcrystals (100 μg sunitinib, 0.04% (w/v) PS80, 0.5% (w/v) methylcellulose) using a 27-gauge needle. After the procedures, antibiotic (neomycin, polymyxin, bacitracin) ointment was applied to prevent possible infection. After 7 days, the growth of blood vessels in the cornea was imaged using a stereoscope (Nikon SMZ) at 10× magnification. The area of neovascularization area was quantified by drawing are along the limbus and the neovascularization area. The area was measured in pixels and quantified by the dividing by the number of pixels in 1 mm² using Adobe Photoshop.

### Histology

Brown Norway rats (6-8 weeks old) were anesthetized and received unilateral subconjunctival injection of either SPC microcrystals (200 μg/5 μL sunitinib, 0.04% PS80, 0.5% methylcellulose) or vehicle (5 μL 0.04% PS80, 0.5% methylcellulose) using a 27-gauge needle. At 1 week and 20 weeks after injection, eyes were enucleated and placed in 4% paraformaldehyde prior to paraffin embedding, sectioning, and H&E staining by the Johns Hopkins Reference Histology Laboratory. Histological sections were evaluated by a board-certified ophthalmic pathologist in a masked manner for the signs of inflammation and cellular damage.

### Statistical Methods

Statistical analyses of two groups were conducted using two-tailed Student's t-test or two-tailed Mann-Whitney test or two-way analysis of variance (ANOVA). For the comparison of multiple groups, one-way ANOVA with Dunnett's multiple comparison test was done. Statistical analysis was done using GraphPad Prism 9. Differences were considered to be statistically significant at a level of p<0.05. For laser-induced ocular hypertension, cumulative IOP exposure was calculated using the trapezoidal rule. Outcome variables were considered to be normally distributed if −0.8<skewness<0.8 and −3.0<kurtosis<3.0. For comparisons of microcrystal rats to sham rats, Student's t-test for 2 independent groups was used for normally distributed outcomes. For outcomes not normally distributed, the Wilcoxon rank sum test for two independent groups was used.

### Results

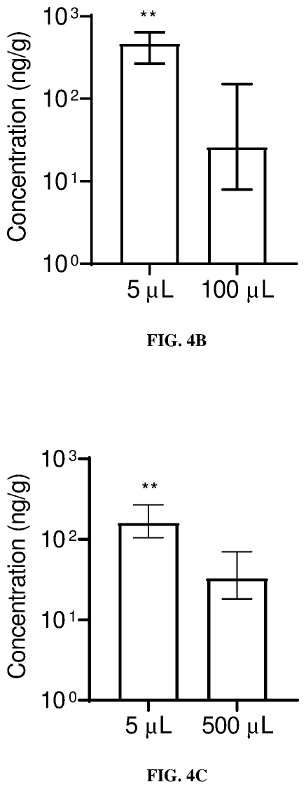

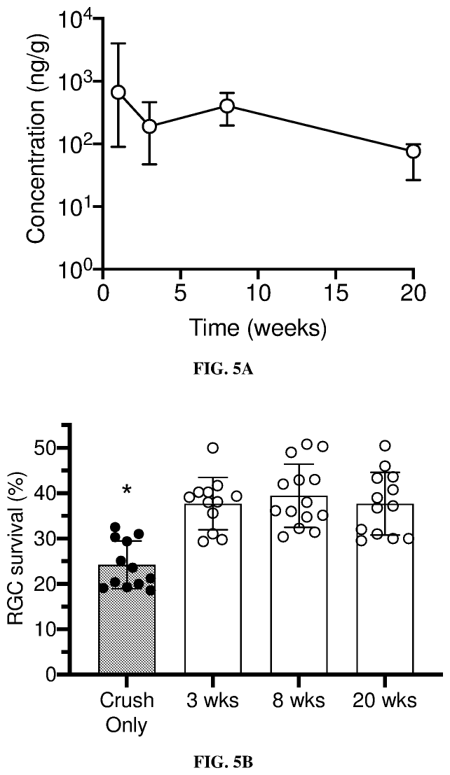

The effect of subconjunctival injection volume on ocular pharmacokinetics was investigated. Since the microcrystals have higher drug loading than typical polymeric microparticles, similar doses were injected to what have been investigated previously for preventing corneal neovascularization with as little as 5 μL (Yang, J., et al., J Control Release, 2020. 327: p. 456-466). Different SPC microcrystal doses were compared to determine a dose that would result in therapeutically relevant sunitinib concentrations in the retina. It was previously found that sunitinib and its major metabolite, N-desethyl sunitinib, had similar neuroprotective activity in both primary mouse RGCs and human stem cell-derived RGCs (Kim, Y. C., et al., Nat Biomed Eng, 2020. 4(11): p. 1053-1062), and thus concentrations were combined for pharmacokinetic analyses. It was found that the 200 μg dose provided significantly higher median retinal combined sunitinib and N-desethyl sunitinib drug concentrations that may be sufficient for protection of RGCs one week after dosing (FIG. 4A). The effect of volume was then investigated when injecting the same sunitinib dose (200 μg), but in 5 μL compared to 100 μL volume in rats. One week after injection, the combined median drug concentration was ˜18-fold higher in the retina for the 5 μL vs. 100 μL injection (FIG. 4B). In rabbits, the combined median drug concentration was ˜4.5-fold higher in the retina when injecting 200 μg of SPC microcrystals in 5 μL vs. 500 μL volume (FIG. 4C). The longitudinal pharmacokinetics was then assessed of the 200 μg sunitinib/5 μL subconjunctival SPC microcrystal dose in rats for up to 20 weeks. The combined drug levels were relatively stable, with a median value of 69.2 ng/g in the retina at 20 weeks (FIG. 5A). Indeed, these drug levels provided by subconjunctival injection of SPC microcrystals were found to be protective of RGCs when performing optic nerve crush after at time points up to 20 weeks. While only 24±5.3% of RGCs survived in untreated control animals, SPC microcrystal treated animals showed a significant increase in RGC survival at 3 weeks (38±5.8%), 8 weeks (39±7.0%) and 20 weeks (38±6.9%) after microcrystal injection (FIG. 5B). Histological assessment of the tissue around the subconjunctival injection site in healthy rats showed an acute mild inflammatory reaction at one week that was resolved before 20 weeks after injection of SPC microcrystals. The retinas of rats receiving subconjunctival injection of SPC microcrystals showed no notable signs of toxicity compared to sham vehicle injection at 1 week.

Next, the SPC microcrystals were tested in a rat model of glaucoma where RGC death and axonal damage is driven by induced IOP elevation, more similar to human disease, rather than mechanical injury. Model induction was consistent across the treated and control groups, as the cumulative IOP elevation was not different between sham and SPC microcrystal treated eyes (Table 1). IOP elevation in this model is associated with increased axial length and width which was not different between treated and sham eyes (Table 1). SPC microcrystal injected eyes had significantly more axonal nerve fibers than sham injected eyes (median of 35,989 for SPC microcrystal injected eyes and 14,418 for sham injected eyes, p=0.04) and a reduced percent axonal loss compared to sham injected eyes (median of 68.8% for SPC microcrystal injected eyes and 87.4% for sham injected eyes, p=0.04).

Table 1. Laser hypertension study outcomes for glaucoma eyes compared to contralateral control eyes. P values shown for comparison between glaucoma eyes from rats treated with SPC microcrystals (SPC) or vehicle (Sham). Values in shaded rows are shown in Figure S8. Student's t-test was used for normally distributed outcomes, and the Wilcoxon rank sum test for two independent groups was used for outcomes not normally distributed.

| | | Mean (SD) or Median | | p- |
|---|---|---|---|---|
| Outcome | Group (n) | (Q1-Q3) | | value |
| Cumulative IOP | SPC (16) | 603.5 | (101.3) | 0.69 |
| (mmHg*day) | Sham (15) | 619.4 | (117.0) | |
| Axial length (mm) | SPC (16) | 6.76 | (0.31) | 0.21 |
| | Sham (15) | 6.63 | (0.28) | |
| Axial width (mm) | SPC (16) | 6.70 | (0.16) | 0.31 |
| | Sham (15) | 6.63 | (0.21) | |
| Nerve density | SPC (16) | 168,980 | (68,720-334,340) | 0.10 |
| (fibers/mm²) | Sham (15) | 86,840 | (47,240-181,000) | |
| Nerve area (mm²) | SPC (16) | 0.21 | (0.02) | 0.08 |
| | Sham (15) | 0.19 | (0.03) | |
| Nerve fibers | SPC (16) | 35,989 | (13,559-75,378) | 0.04 |
| | Sham (15) | 14,418 | (8,899-29,921) | |
| Nerve diameter (mm) | SPC (16) | 0.32 | (0.06) | 0.39 |
| | Sham (15) | 0.30 | (0.05) | |
| Axon loss (%) | SPC (16) | 68.8 | (34.6-88.3) | 0.04 |
| | Sham (15) | 87.4 | (73.7-92.2) | |
| RGC loss (%) | SPC (7) | 63.3 | (28.0) | 0.08 |
| | Sham (12) | 81.3 | (15.0) | |

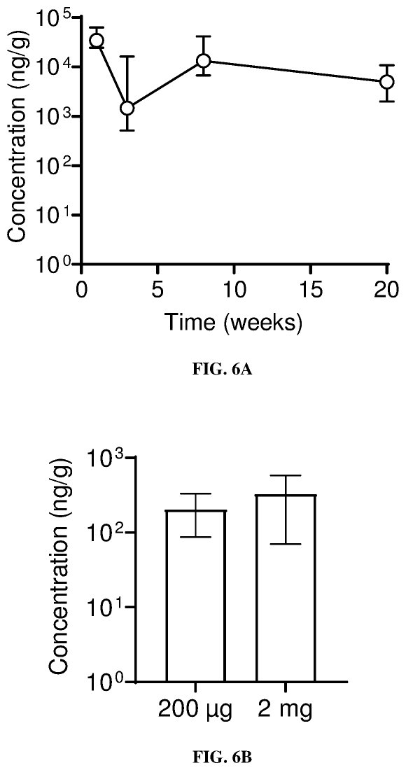

It was then sought to determine whether therapeutically relevant drug concentrations could be achieved with subconjunctival injection of SPC microcrystals in an animal with larger eyes and thicker sclera, such as pigs. Because of the larger size, the 200 μg dose and a larger dose of 2 mg were tested. The median combined retinal drug concentration observed in pigs at 7 days after injecting the 2 mg dose (11.1 ng/g) was not statistically different than the concentrations that maintained efficacy in protecting RGCs in rats at 20 weeks in this study (FIG. 5A), and similar to effective concentrations observed in a separate study using an eye drop formulation (˜25.6 ng/g). The minimum effective concentration of sunitinib for protecting RGCs is not known, though future studies could include further dose ranging studies to achieve higher retina drug concentrations in pigs and evaluate the duration. Similar to what was observed in the longitudinal study in rats (FIG. 6A), combined drug levels in the choroid and retinal pigment epithelium (Ch/RPE) were ˜10-fold higher than levels in the retina in pigs (FIG. 6B), likely indicative of the known melanin binding properties of sunitinib.

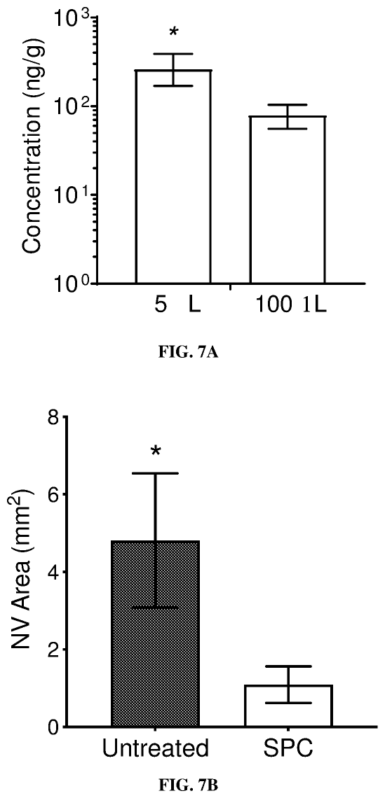

Sunitinib was developed as a tyrosine kinase inhibitor and is better known for its antiangiogenic properties via inhibition of vascular endothelial growth factor receptors (VEGFR) and platelet-derived growth factor receptors (PDGFR) (Roskoski, R., Jr., Biochem Biophys Res Commun, 2007. 356(2): p. 323-8). It was previously demonstrated that subconjunctival injection of polymeric microparticles to deliver sunitinib malate provided inhibition of corneal neovascularization in rats, whereas injection of free sunitinib malate in solution had no effect on neovascularization (Yang, J., et al., J Control Release, 2020. 327: p. 456-466). Thus, it was then tested whether anterior subconjunctival injection of SPC microcrystals inhibited corneal neovascularization. Similar to what was observed for the retina, subconjunctival injection of 100 μg dose of SPC microcrystals in a 5 μL volume resulted in increased median drug concentrations in the cornea (321 ng/g) at 7 days after injection compared to a 100 μg dose injected in a 100 μL volume (96.3 ng/g) (FIG. 7A). When 100 μg of SPC microcrystals was administered by subconjunctival injection in a 5 μL volume in a rat model of induced corneal neovascularization, the area of new blood vessel growth was reduced by 4.4-fold after 7 days compared to untreated rats (1.1±0.2 mm² vs. 4.8±0.6 mm²) (FIG. 7B).

While elevated IOP is an important risk factor implicated in the onset and progression of glaucoma, studies have shown that disease progression continues in some patients despite IOP lowering (Nucci, C., et al., Eye (Lond), 2018. 32(5): p. 938-945). Thus, efforts have been underway to develop molecules that promote the survival of RGCs independent of IOP, which may be used as a complementary therapy to standard IOP lowering regimens. Many glaucoma patients are prescribed complex eye drop regimens that may require dosing multiple times per day as well as multiple types of drops, which leads to issues with adherence (Nordstrom, B. L., et al., Am J Ophthalmol, 2005. 140(4): p. 598-606; Stewart, W. C., et al., Am J Ophthalmol, 1993. 116(2): p. 176-81; Schwartz, G. F. and H. A. Quigley, Surv Ophthalmol, 2008. 53 Suppl1: p. S57-68). Thus, an injectable sustained-release strategy may be attractive for achieving long-lasting neuroprotection in glaucoma patients. Here, we demonstrated that a single subconjunctival injection of SPC microcrystals provided sustained neuroprotection in a rat optic nerve crush model for at least 20 weeks.

Among the various periocular injections, subconjunctival injection is considered as one of the safer ways to administer drugs because the tip of the needle is visible throughout the procedure, thereby minimizing the risk associated with hemorrhage or perforation of the eye (Morgan, C. M., et al., Ophthalmology, 1988. 95(5): p. 660-665; Hay, A., et al., Ophthalmology, 1991. 98(7): p. 1017-1024; Jain, V. K., et al., Ophthalmic Surgery and Lasers, 1991. 22(9): p. 508-511). Further, subconjunctival injection of steroids was shown to provide higher intraocular bioavailability with lower systemic absorption when compared to peribulbar or oral administration (Weijtens, O., et al., Ophthalmology, 2002. 109(10): p. 1887-91; Weijtens, O., et al., Am J Ophthalmol, 1999. 128(2): p. 192-7). Subconjunctival injection of water-soluble drugs leads to increased intraocular bioavailability, potentially due to the higher concentration gradient and increased transscleral drug penetration (Pan, Q., et al., J Control Release, 2015. 201: p. 32-40; Prausnitz, M. R. and J. S. Noonan, J Pharm Sci, 1998. 87(12): p. 1479-88; Thakur, A., R. S. Kadam, and U. B. Kompella, Drug Metab Dispos, 2011. 39(5): p. 771-81). However, water-soluble drugs are cleared from the subconjunctival space within hours limiting the duration of therapeutic effect that can be achieved. Thus, a subconjunctival drug delivery formulation would ideally encapsulate and provide sustained release of water-soluble drugs, which is generally difficult to achieve. Other approaches were previously described for loading water-soluble dexamethasone sodium phosphate and sunitinib malate into polymeric particles for subconjunctival injection, providing sustained prevention or treatment of corneal graft rejection (Pan, Q., et al., J Control Release, 2015. 201: p. 32-40), corneal neovascularization (Yang, J., et al., J Control Release, 2020. 327: p. 456-466; Wang, B., et al., Nanomedicine, 2019. 17: p. 119-123) and uveitis (Luo, L., et al., J Control Release, 2019. 296: p. 68-80). However, one potential drawback was relatively low drug loading in the range of 6-8% by weight. Here, the 1:1 molar ratio of pamoate and sunitinib in the SPC microcrystals means that the drug loading was ˜50%. Higher drug loading allows for smaller injection volumes, which here, resulted in increased drug delivery to the posterior segment. It is possible that the lower volume, and thus, lower surface area for drug absorption, decreased drug clearance via conjunctival blood vessels and lymphatic drainage (Kim, H., et al., Investigative Ophthalmology & Visual Science, 2004. 45(8): p. 2722-2731; Robinson, M. R., et al., Exp Eye Res, 2006. 82(3): p. 479-87). Smaller injection volumes may also be attractive for cosmetic reasons and may reduce the potential for tissue fibrosis with repeated injections over the duration of glaucoma treatment, which may last many years or even decades.

As with other modes of ocular administration, the choice of animal model is important when evaluating drug delivery to the posterior segment with subconjunctival injection. Mice and rats are often useful in terms of availability of disease models that can be used to demonstrate therapeutic efficacy and determine the range of drug concentrations that are effective. However, to better predict whether drug could be delivered at therapeutically relevant concentrations in human eyes, larger animals must often be used. Compared to rodents and rabbits, pigs have ocular structural features that may more closely recapitulate the human eye. For example, the pig scleral thickness range of 0.3-0.8 mm is similar to the range of thickness reported for humans (0.4-0.9 mm), as is the choroidal blood flow rate (˜1.6 m/min for pigs and 1.4 m/min for humans) (Schopf, L. R., et al., Transl Vis Sci Technol, 2015. 4(3): p. 11; Vurgese, S., S. Panda-Jonas, and J. B. Jonas, PLoS One, 2012. 7(1): p. e29692; Tomquist, P., A. Alm, and A. Bill, Acta Physiol Scand, 1979. 106(3): p. 343-50; Zhang, Y., et al., Doc Ophthalmol, 2013. 126(3): p. 187-97). In contrast, scleral thickness in mice and rats is much thinner, 0.04 mm and <0.1 mm, respectively (Cone-Kimball, E., et al., Mol Vis, 2013. 19: p. 2023-39; Girard, M. J., et al., Invest Ophthalmol Vis Sci, 2011. 52(13): p. 9684-93). Thus, it is not surprising that lower drug levels in the retina were observed when injecting the same dose of SPC microcrystals of pigs compared to rats. A ten-fold higher dose in pigs resulted in retinal drug concentrations that reached the range of that was observed to be effective in protecting RGCs in rats. Next steps in development of the SPC microcrystal formulation would include additional dose ranging and longitudinal pharmacokinetic studies in pigs.

Another factor that can affect drug pharmacokinetics and pharmacodynamics is the binding of drugs to ocular melanin. It has been demonstrated that drug binding to melanin in the sclera can reduce the amount that is able to reach the posterior segment after subconjunctival injection (Cheruvu, N. P., A. C. Amrite, and U. B. Kompella, Invest Ophthalmol Vis Sci, 2008. 49(1): p. 333-41; Rimpela, A. K., et al., Adv Drug Deliv Rev, 2018. 126: p. 23-43; Jakubiak, P., et al., Mol Pharm, 2019. 16(12): p. 4890-4901). In a separate study using an eye drop formulation, it has been demonstrated that binding of sunitinib to melanin in the choroid and retinal pigment epithelium (Ch/RPE) appeared to be beneficial for increasing the amount drug that reached the retina. Thus, for drugs that naturally bind to melanin, such as sunitinib, the use of pigmented animals is important. However, this can be an issue when disease models require the use of albino animals, such as was the case with the induced ocular hypertension model described here (Johnson, T. V. and S. I. Tomarev, Brain Res Bull, 2010. 81(2-3): p. 349-58). In this model, the laser-induced photocoagulation of the trabecular meshwork is negatively impacted by increased pigmentation in the trabecular meshwork, which limits the potential for induced IOP elevation (Ishikawa, M., et al., Biomed Res Int, 2015. 2015: p. 281214). With subconjunctival injection in albino animals, the benefit of reduced binding of drug to melanin in the sclera may be offset by the reduced binding in the choroid/RPE, leading to less effective delivery to the RGCs. In contrast, the optic nerve crush model used here is a mechanical model of RGC injury that has similar effects in albino and pigmented rats. In pigmented rats, a single subconjunctival injection of 200 μg SPC microcrystals provided sustained retina drug concentrations and protection of RGCs for at least 20 weeks.

Many FDA approved drugs have moderate to high water solubility through ionization and formulation as a pharmaceutical salt to enhance biodistribution (Sasaki, H., et al., Delivery of drugs to the eye by topical application. Progress in Retinal and Eye Research, 1996. 15(2): p. 583-620; Prausnitz, M. R. and J. S. Noonan, Journal of Pharmaceutical Sciences, 1998. 87(12): p. 1479-1488)]. The approach described here for ionic complexation to reversibly lower the water solubility for formulation for sustained release could be applied to numerous other pharmaceuticals. The SPC microcrystal formulation has high translational potential, as oral sunitinib malate (SUTENT®) is FDA-approved for treatment of some cancers. Further, an intravitreal polymeric microparticle formulation that provides sustained intraocular delivery of sunitinib malate is in Phase 2 clinical trials for neovascular age-related macular degeneration (ClinicalTrials.gov Identifier: NCT03953079) (A Depot Formulation of Sunitinib Malate (GB-102) Compared to Aflibercept in Subjects With Wet AMD (ALTISSIMO) [cited 2020 Dec. 26th]) and macular edema (ClinicalTrials.gov Identifier: NCT04085341). Pamoic acid has a history of safety as a counterion used in several oral drug formulations, though thorough safety studies will be required to demonstrate safety with subconjunctival injection. A safe and efficacious injectable formulation with the potential for twice yearly administration for neuroprotection in glaucoma would likely have a significant impact in helping to maintain vision and quality of life.

In this study, an approach for formulating sunitinib for prolonged therapeutic effects with subconjunctival injection has describe. Sunitinib is highly water soluble in the pharmaceutical salt form, which presents a challenge for formulation to achieve sustained release. However, it was previously observed that the water-soluble salt form is preferred for achieving intraocular drug penetration upon subconjunctival injection. To reduce the solubility of sunitinib malate for formulation purposes, hydrophobic, water soluble counterion salts were screened for the ability to form an insoluble complex. Then, rather than load the insoluble complex into a polymer matrix particle, crystalline drug microcrystals with high drug loading were formulated. This approach allows formulating water insoluble ion-complex microcrystals containing ˜50% sunitinib by mass. It has been demonstrated that a single subconjunctival injection of sunitinib-pamoate complex (SPC) microcrystals provided at least 20 weeks of RGC protection in a rat model of optic nerve crush. As sunitinib also inhibits blood vessel growth, subconjunctival injection of SPC microcrystals inhibited vessel growth in a rat model of corneal neovascularization. Importantly, therapeutically relevant retina drug concentrations were achieved with subconjunctival injection of SPC microcrystals in pigs.

Variations and modifications of the foregoing will be obvious to those skilled in the art and are intended to come within the scope of the appended claims.

1. An injectable formulation for administration to the eye, comprising
(a) one or more therapeutic, prophylactic or diagnostic agents in the form of microcrystals; and
(b) one or more pharmaceutically acceptable excipients for administration into the eye.

2. The injectable formulation of claim 1, wherein the one or more therapeutic agents, prophylactic agents, or diagnostic agents bind to a ligand or receptor in or on the eye.

3. The injectable formulation of claim 2, wherein the one or more therapeutic agents is an inhibitor of dual leucine zipper kinase (DLK).

4. The injectable formulation of claim 2, wherein the one or more therapeutic agents, prophylactic agents, and/or diagnostic agents bind to melanin with a dissociation constant (K_{d}) of less than 1×10⁻³ M, 1×10⁻⁴ M, 1×10⁻⁵ M, or 1×10⁻⁶ M.

5. The injectable formulation of claim 2, wherein the one or more therapeutic agents or prophylactic agents is sunitinib, acriflavine, or derivative, analogue, or prodrug thereof.

6. The injectable formulation of claim 2, wherein the microcrystals are formed of sunitinib and pamoic acid.

7. The injectable formulation of claim 1, wherein the size of the microcrystals is between about 0.1 microns and about 500 microns.

8. The injectable formulation of claim 1, wherein the formulation has a pH between 6 and 8.

9. The injectable formulation of claim 1, formulated for intravitreal, subconjunctival, or suprachoroidal administration.

10. The injectable formulation of claim 1, wherein the formulation does not contain a preservative or surfactant.

11. The injectable formulation of claim 1, wherein the volume of formulation is between about 0.1 μl and about 100 μl.

12. The injectable formulation of claim 1, wherein the one or more active agents in the microcrystals are in an amount effective to provide therapeutic or prophylactic efficacy for at least six months or longer at or around the site of application in the eye.

13. A method for preventing and treating one or more diseases, conditions, or injuries of the eye in a subject comprising administering to the eye of the subject a therapeutically effective amount of the formulation of claim 1.

14. The method of claim 13, wherein the one or more diseases, conditions, and injuries of the eye are diseases of the posterior segment of the eye.

15. The method of claim 13, wherein the one or more diseases, conditions, and injuries of the eye are disease of the retina, choroid, and/or optic nerve.

16. The method of claim 13, wherein the one or more diseases, conditions, and injuries of the eye are selected from the group consisting of retinal tear, retinal detachment, diabetic retinopathy, epiretinal membrane, macular hole, macular degeneration, retinitis pigmentosa, retinal neovascularization, and choroid neovascularization.

17. The method of claim 13, wherein the formulation delivers an effective amount of microcrystals of therapeutic agent to the choroid and/or retinal pigmented epithelium (RPE) in the eye to reduce retinal and/or choroidal neovascularization.

18. The method of claim 17, wherein the amount of therapeutic agent(s) administered as microcrystals is effective to reduce retinal and/or choroidal neovascularization by 10%, 20%, 30%, 40%, 50%, or more than 50% of the equivalent amount of the same active agent(s) delivered not in the form of microcrystals.

19. The method of claim 13, wherein the formulation is administered via intravitreal, subconjunctival, or suprachoroidal injection.

20. The method of claim 13, wherein the formulation is administered once a week or less frequently.

21. The method of claim 13, wherein the method provides therapeutic or prophylactic efficacy in the subject following a single administration for a period of time selected from one week, two weeks, three weeks, one month, two months, three months, four months, five months, six months, eight months, ten months, and one year.

22. The injectable formulation of claim 7 wherein the size of the microcrystals is between about 1 micron and about 250 microns.

23. The injectable formulation of claim 11, wherein the volume of formulation is between about 1 μl and 50 μl.
